(19) United States
(12) Patent Application Publication
(10) Pub. No.: US 2022/0105295 A1
(43) Pub. Date: Apr. 7, 2022

(54) Nasal Cannula Holding Assembly

(71) Applicant: Ralph Thompson, Anacoco, LA (US)

(72) Inventor: Ralph Thompson, Anacoco, LA (US)

(21) Appl. No.: 17/061,633

(22) Filed: Oct. 2, 2020

(51) Int. Cl. A61M 16/06

(52) U.S. Cl. CPC A61M 16/0683; A61M 2209/08; A61M 16/0666

(57) ABSTRACT

A nasal cannula holding assembly includes a pair of straps that each has a plurality of first holes extending therethrough and a plurality of second holes extending therethrough. Each of the straps includes a plurality of first engagements and a plurality of second engagements. Each of the first engagements engages a respective one of the first holes and each of the second engagements engages a respective one of the second holes. In this way a first loop and a second loop are defined on the straps to define a second loop thereby facilitating an oxygen tube of the nasal cannula to be extended therethrough. Thus, the oxygen tube is inhibited from contacting the user's eyes when the nasal cannula is worn.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to holding devices and more particularly pertains to a new holding device for inhibiting an oxygen tube of a nasal cannula from touching a user's eyes when the nasal cannula is worn.

### (2) Description of Related Art Including Information Disclosed Under 37 CFR 1.97 and 1.98

The prior art relates to holding devices including a variety of holding devices that comprise a band that can be wrapped around user's head for securing an oxygen hose. The prior art discloses a holding device that is attached to an oxygen hose of a nasal cannula such that the oxygen hose is routed behind the user's head.

### BRIEF SUMMARY OF THE INVENTION

An embodiment of the disclosure meets the needs presented above by generally comprising a pair of straps that each has a plurality of first holes extending therethrough and a plurality of second holes extending therethrough. Each of the straps includes a plurality of first engagements and a plurality of second engagements. Each of the first engagements engages a respective one of the first holes and each of the second engagements engages a respective one of the second holes. In this way a first loop and a second loop are defined on the straps to define a second loop thereby facilitating an oxygen tube of the nasal cannula to be extended therethrough. Thus, the oxygen tube is inhibited from contacting the user's eyes when the nasal cannula is worn.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a nasal cannula holding assembly according to an embodiment of the disclosure.

FIG. 2 is a perspective view of an embodiment of the disclosure showing a first loop and a second loop formed into a strap.

FIG. 3 is a top view of an embodiment of the disclosure.

FIG. 4 is a right side view of an embodiment of the disclosure.

FIG. 5 is a bottom view of an embodiment of the disclosure.

FIG. 6 is a front perspective in-use view of an embodiment of the disclosure.

FIG. 7 is a left side perspective in-use view of an embodiment of the disclosure.

### DETAILED DESCRIPTION OF THE INVENTION

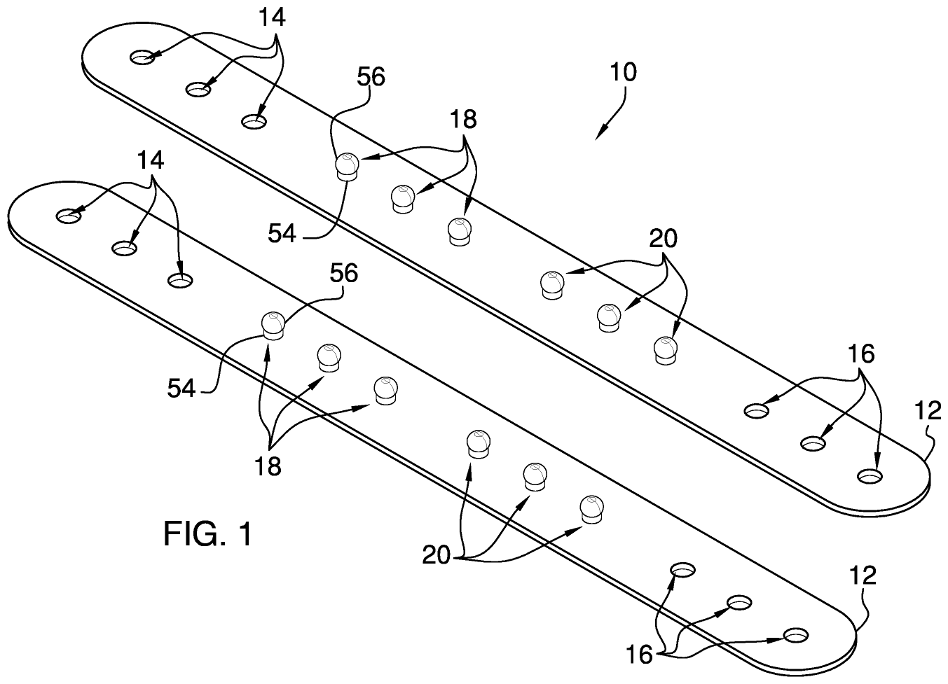

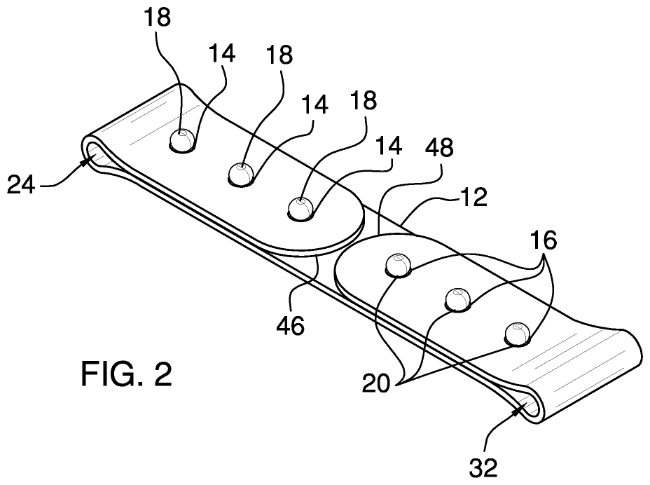

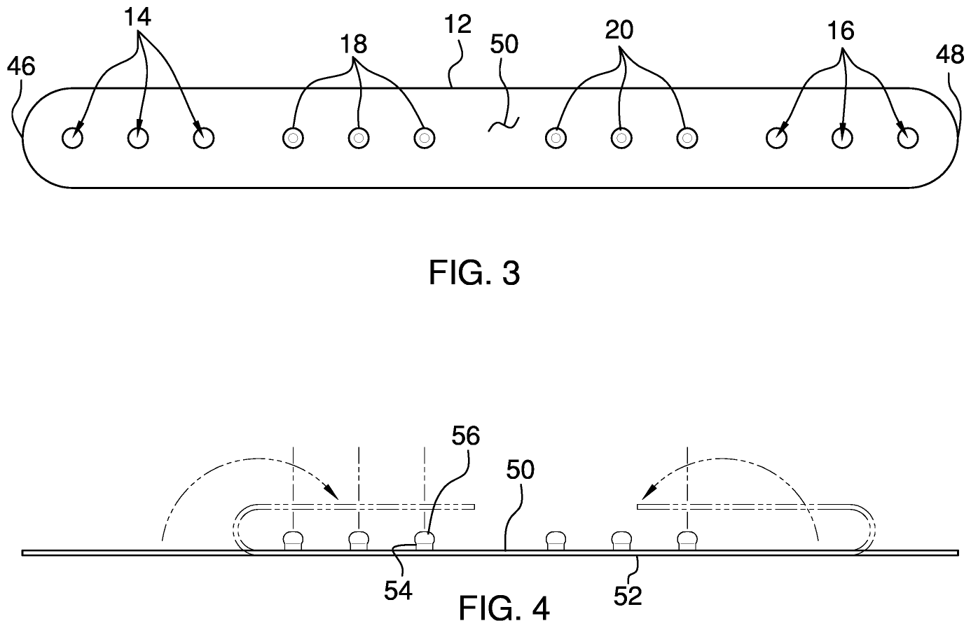

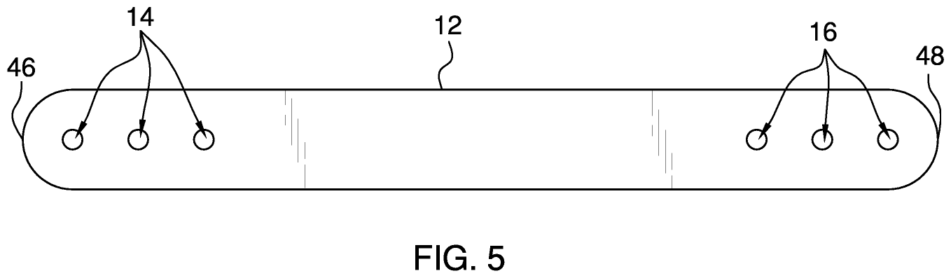

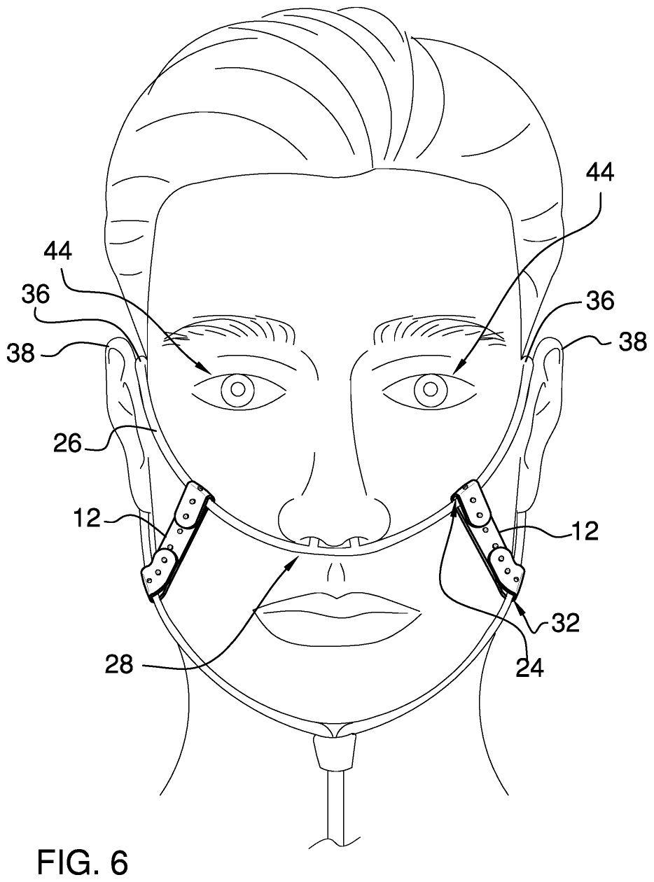

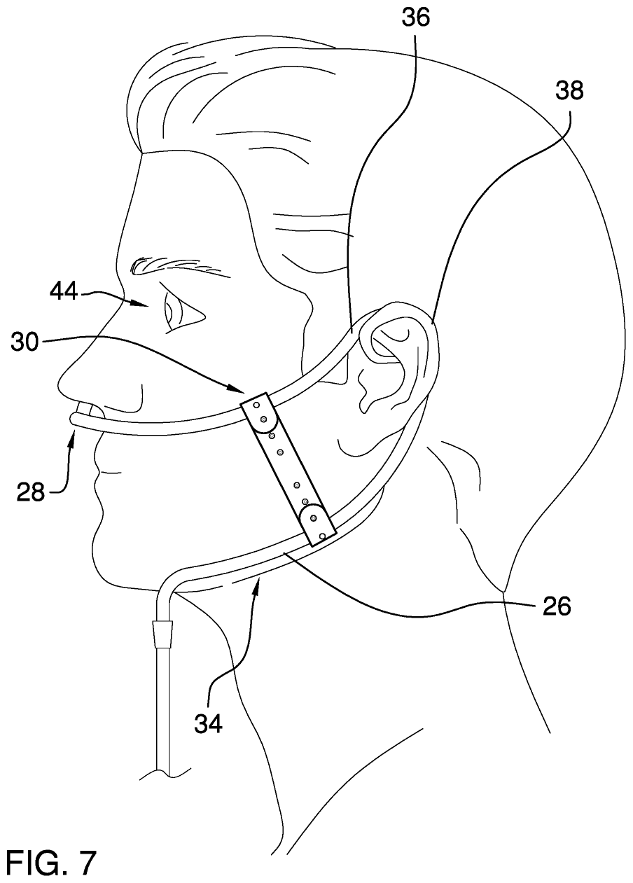

With reference now to the drawings, and in particular to FIGS. 1 through 7 thereof, a new holding device embodying the principles and concepts of an embodiment of the disclosure and generally designated by the reference numeral 10 will be described.

As best illustrated in FIGS. 1 through 7, the nasal cannula holding assembly 10 generally comprises a pair of straps 12 that each has a plurality of first holes 14 extending therethrough and a plurality of second holes 16 extending therethrough. Each of the straps 12 includes a plurality of first engagements 18 and a plurality of second engagements 20. Each of the first engagements 18 on a respective one of the straps 12 engages a respective one of the first holes 14 on the respective strap 12 to define a first loop 24 on the respective strap 12. In this way an oxygen tube 26 of a nasal cannula 28 can be extended through the first loop 24 such that the first loop 24 is positioned on the user's cheek 30. Moreover, each of the second engagements 20 on a respective one of the straps 12 engages a respective one of the second holes 16 on the respective strap 12 to define a second loop 32 on the respective strap 12. In this way the oxygen tube 26 of the nasal cannula 28 can be extended through the second loop 32 such that the second loop 32 is positioned on the user's jaw line 34.

Each of the first loop 24 and the second loop 32 defines a loop 36 on the oxygen tube 26 when the oxygen tube 26 is extended through each of the first loop 24 and the second loop 32. Additionally, the loop 36 on the oxygen tube 26 defined by each of the straps 22 can be positioned around a respective one of the user's ears 38. In this way each of the straps 22 is positioned on opposite sides of the user's face 42 when the nasal cannula 28 is worn. Thus, the straps 22 inhibit the oxygen tube 26 from contacting the user's eyes 44 and subsequently causing discomfort for the user when the nasal cannula 28 is worn.

Each of the straps 12 has a first end 46, a second end 48, a top surface 50 and a bottom surface 52. Each of the straps 12 is elongated between the first end 46 and the second end 48 to a length of at least 6.0 inches. Each of the first holes 14 extends through the top surface 50 and the bottom surface 52. Additionally, the first holes 14 are spaced apart from each other and are distributed from the first end 46 toward the second end 48. Each of the second holes 16 extends through the top surface 50 and the bottom surface 52. Additionally, the second holes 16 are spaced apart from each other and are distributed from the second end 48 toward the first end 46.

Each of the first engagements 18 is coupled to the top surface 50. Each of the first engagements 18 is spaced apart from each other and is distributed between the first holes 14 and a center point 53 of a respective strap 12. Each of the second engagements 20 is spaced apart from each other and is distributed between the second holes 16 and the center point 53 of a respective strap 12. Additionally, each of the first engagements 18 and each of the second engagements 20 comprise a stem 54 extending upwardly from the top surface 50 and a ball 56 positioned on the stem 54 such that the ball 56 is spaced from the top surface 50. The ball 56 on each of the first engagements 18 and each of the second engagements 20 extends through the respective first hole 14 or the respective second hole 16. Moreover, the ball 56 on each of the first engagements 18 and each of the second engagements 20 has a diameter that is greater than a diameter of any of the first holes 14 or the second holes 16. Each of the straps 22 is comprised of a resiliently deformable material thereby facilitating the straps 22 to be stretched around the ball 56 on each of the first engagements 18 and each of the second engagements 20 when the ball 56 on each of the first engagements 18 and each of the second engagements 20 is extended through the respective first hole 14 or the respective second hole 16.

In use, the first loop 24 and the second loop 32 are formed on each of the straps 22 thereby facilitating the oxygen tube 26 to be extended through the first loop 24 and the second loop 32 on each of the straps 22. Each of the first loop 24 and the second loop 32 defines a loop 36 on the oxygen tube 26 when the oxygen tube 26 is extended through each of the first loop 24 and the second loop 32. Additionally, the loop 36 on the oxygen tube 26 defined by each of the straps 22 can be positioned around a respective one of the user's ears 38. In this way each of the straps 22 is positioned on opposite sides of the user's face 42 when the nasal cannula 28 is worn. Thus, the straps 22 inhibit the oxygen tube 26 from contacting the user's eyes 44 and subsequently causing discomfort for the user when the nasal cannula 28 is worn.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A nasal cannula holding assembly for forming an oxygen tube into a loop around a user's ear thereby inhibiting the oxygen tube from riding up on the user's face and irritating the user's eyes, said assembly comprising:
a pair of straps, each of said straps having a plurality of first holes extending therethrough and a plurality of second holes extending therethrough, each of said straps including a plurality of first engagements and a plurality of second engagements, each of said first engagements on a respective one of said straps engaging a respective one of said first holes on said respective strap to define a first loop on said respective strap thereby facilitating said first loop on each of said straps to have an oxygen tube of a nasal cannula extended therethrough wherein said first loop is configured to be positioned on the user's cheek, each of said second engagements on a respective one of said straps engaging a respective one of said second holes on said respective strap to define a second loop on said respective strap thereby facilitating said second loop on each of said straps to have the oxygen tube of the nasal cannula extended therethrough wherein said second loop is configured to be positioned on the user's jaw line, each of said first loop and said second loop defining a loop on the oxygen tube when the oxygen tube is extended through each of said first loop and said second loop wherein the loop on the oxygen tube is configured to be positioned around a respective one of the user's ears.

2. The assembly according to claim 1, wherein each of said straps has a first end, a second end, a top surface and a bottom surface, each of said straps being elongated between said first end and said second end, each of said first holes extending through said top surface and said bottom surface, said first holes being spaced apart from each other and being distributed from said first end toward said second end.

3. The assembly according to claim 2, wherein each of said second holes extends through said top surface and said bottom surface, each of said second holes being spaced apart from each other and being distributed from said second end toward said first end.

4. The assembly according to claim 2, wherein each of said first engagements is coupled to said top surface, each of said first engagements being spaced apart from each other and being distributed between said first holes and a center point of a respective strap.

5. The assembly according to claim 2, wherein each of said second engagements is spaced apart from each other and is distributed between said second holes and said center point of a respective strap.

6. The assembly according to claim 2, wherein each of said first engagements and each of said second engagements comprises a stem extending upwardly from said top surface and a ball positioned on said stem such that said ball is spaced from said top surface.

7. The assembly according to claim 6, wherein said ball on each of said first engagements and each of said second engagements extends through said respective first hole or said respective second hole, said ball on each of said first engagements and each of said second engagements having a diameter being greater than a diameter of any of said first holes or said second holes.

8. The assembly according to claim 7, wherein said strap is comprised of a resiliently deformable material thereby facilitating said strap to be stretched around said ball on each of said first engagements and each of said second engagements when said ball on each of said first engagements and each of said second engagements is extended through said respective first hole or said respective second hole.

9. A nasal cannula holding assembly for forming an oxygen tube into a loop around a user's ear thereby inhibiting the oxygen tube from riding up on the user's face and irritating the user's eyes, said assembly comprising:
a pair of straps, each of said straps having a plurality of first holes extending therethrough and a plurality of second holes extending therethrough, each of said straps including a plurality of first engagements and a plurality of second engagements, each of said first engagements on a respective one of said straps engaging a respective one of said first holes on said respective strap to define a first loop on said respective strap thereby facilitating said first loop on each of said straps to have an oxygen tube of a nasal cannula extended therethrough wherein said first loop is configured to be positioned on the user's cheek, each of said second engagements on a respective one of said straps engaging a respective one of said second holes on said respective strap to define a second loop on said respective strap thereby facilitating said second loop on each of said straps to have the oxygen tube of the nasal cannula extended therethrough wherein said second loop is configured to be positioned on the user's jaw line, each of said first loop and said second loop defining a loop on the oxygen tube when the oxygen tube is extended through each of said first loop and said second loop wherein the loop on the oxygen tube is configured to be positioned around a respective one of the user's ears, each of said straps having a first end, a second end, a top surface and a bottom surface, each of said straps being elongated between said first end and said second end, each of said first holes extending through said top surface and said bottom surface, said first holes being spaced apart from each other and being distributed from said first end toward said second end, each of said second holes extending through said top surface and said bottom surface, each of said second holes being spaced apart from each other and being distributed from said second end toward said first end, each of said first engagements being coupled to said top surface, each of said first engagements being spaced apart from each other and being distributed between said first holes and a center point of a respective strap, each of said second engagements being spaced apart from each other and being distributed between said second holes and said center point of a respective strap, each of said first engagements and each of said second engagements comprising a stem extending upwardly from said top surface and a ball positioned on said stem such that said ball is spaced from said top surface, said ball on each of said first engagements and each of said second engagements extending through said respective first hole or said respective second hole, said ball on each of said first engagements and each of said second engagements having a diameter being greater than a diameter of any of said first holes or said second holes, said strap being comprised of a resiliently deformable material thereby facilitating said strap to be stretched around said ball on each of said first engagements and each of said second engagements when said ball on each of said first engagements and each of said second engagements is extended through said respective first hole or said respective second hole.
